(19) United States
(12) Patent Application Publication
(10) Pub. No.: US 2016/0101067 A1
(43) Pub. Date: Apr. 14, 2016

(54) SUPRAMOLECULAR COMPLEXES OF POLYANIONIC POLYMERS AND SPERMIDINE IN TISSUE MAINTENANCE AND REPAIR

(71) Applicant: TIXUPHARMA, Milan (IT)

(72) Inventor: Carlo GHISALBERTI, Sao Paulo (BR)

(21) Appl. No.: 14/973,135

(22) Filed: Dec. 17, 2015

Related U.S. Application Data
(63) Continuation of application No. 13/813,939, filed on Aug. 9, 2013.

(51) Int. Cl. A61K 31/132; A61K 47/48

(52) U.S. Cl. CPC A61K 31/132; A61K 47/4823; A61K 47/48192

(57) ABSTRACT

Supramolecular complexes are formed by polyanionic polymers and spermidine having a ratio of anionic equivalents ranging from 10¹:1 to 10⁷:1 eq/eq, more preferably from 10²:1 to 10⁴:1 eq/eq, whose components are linked by non-covalent, ionic interactions. The supramolecular complexes are exhibit high potency in eliciting fibroblast proliferation. Disclosed are medicinal/cosmetic compositions containing the supramolecular complexes for the trophism, maintenance, regeneration, and repair of connective tissues and mucosae in damaged or senescent conditions.

### CROSS REFERENCE

This application is a continuation of application Ser. No. 13/813,939, filed Aug. 9, 2013, pending, which is the U.S. National Phase of International Application No. PCT/IB2011/001771 filed Aug. 1, 2011 which designated in the U.S. and claims priority to Italian Application Nos. MI2010A001491 filed Aug. 4, 2010, MI2010A002277 filed Dec. 14, 2010, and MI2010A002308 filed Dec. 16, 2010, the entire contents of each of which are hereby incorporated by reference.

### FIELD OF THE INVENTION

The invention refers to supramolecular complexes formed by polyanionic polymers and spermidine; and to medicinal/cosmetic compositions comprising them for the treatment, maintenance or repair of connective tissue and mucosae.

### BACKGROUND

Spermidine belongs to the group of polyamines (PA), metabolic polycations that link negatively charged DNA, RNA, proteins, phospholipids, and nucleoside triphosphates.

This ability could explain a contribution in cell proliferation and differentiation, see Heby O. Differentiation 1981; 19:1-20; and Cohen S S. A Guide to the Polyamines. New York: Oxford University Press; 1998; 185-230.

Nevertheless their biological role is difficult to be clearly defined. PA are in fact required for transcription of the proto-oncogenes c-myc and c-fos. In particular, spermidine preferentially stimulates the transcription and the expression of c-myc, while putrescine of c-fos (Tabib & Bachrach Int J Biochem Cell Biol 1999; 31:1289-95). A role in transduction of signals between cell membrane and nucleus was previously found (Tabib & Bachrach. Biochem Biophys Res Commun 1994; 202:720-7). Spermidine is also involved in TGF-6 signal transduction (Blachowski S et al., Int J Biochem 1994; 26:891-897) and seems to be necessary for normal expression of the TGF-β gene during cell migration.

Several patents claim spermidine and other PA in various therapeutic contexts, namely WO97/014415 and WO99/051213 for local analgesia and eczema; U.S. Pat. No. 6,555,140 to increase male fertility and libido; U.S. Pat. No. 06,252,838 as skin anti-aging; U.S. Pat. No. 04,242,701 to treat alcoholism; WO9852552 as anti-cancer; and U.S. Pat. No. 5,432,202 as anti-hypertensive Ca-antagonists.

A typical behaviour of spermidine is the interaction with anionic macromolecules to produce supramolecular complexes, which for example modulate the enzyme/DNA interactions (Isobe H et al. Chem Commun (Camb). 2005; 28; (12):1549-51).

Supramolecular complexes formed by spermidine and phosphate groups change the condensation status of DNA, and protect it from the nucleases activities (D′Agostino L et al. IUBMB Life. 2006; 58(2):75-82). A similar property was found on the spermidine supramolecular, self-assembled aggregates of the polyamine reportedly behaving as protecting factors (D′Agostino L et al. FEBS J. 2009;276(8): 2324-35).

WO2010049562 discloses the supramolecular complexes of polyanionic polymer with spermidine or spermine by ratios comprised between 1:0.1 and 1:05 w/w, that clearly approach the equimolarity. Their purported use is the delivery of other bioactive agents.

The only known application are the anti-alopecia products marketed as Bioscalin® by Giuliani SpA (Milan, Italy), with spermidine HCl branded Biogenina and Cronobiogenina. Formulations and anti-alopecia methods based on spermidine HCl have been patented by Giuliani in EP1469843, WO03063851, and WO2010060729.

While there is a clear need for therapeutics capable to promote tissue regeneration by means of cell proliferation, the design of spermidine complexes having high efficiency in this specific issue has never been attempted so far.

### SUMMARY OF THE INVENTION

It was surprisingly discovered that certain supramolecular complexes formed by polyanionic polymers and spermidine have high efficiency in connective regeneration.

In an aspect, the invention refers to supramolecular complexes characterized by ratios of anionic equivalents (of polyanionic polymer) and cationic equivalents (of spermidine) from 10:1 to 10⁷:1 eq/eq, further characterized by high efficacy in regenerative treatments.

In another aspect, the inventive supramolecular complexes have preferred ratios from 10²:1 to 10⁴:1 eq/eq.

In still another aspect, the invention refers to compositions comprising the aforesaid supramolecular complexes for the maintenance and repair of damaged or senescent tissues.

These and other features of the invention are best described herein after.

### BRIEF DESCRIPTION OF THE DRAWINGS

FIG. 1 shows the % of fibroblasts increase after treatment with 10:1, 10²:1, 10³:1, 10⁴:1, 10⁵:1, and 10⁶:1 eq/eq HA-spermidine complexes at 24 h (light shaded) and 48 h (darker shaded) vs untreated cell; first 2 bars is the HA-spermidine 1:1 eq/eq complex (stripped bars, outside invention); last 2 bars is pure HA 0.1% w/v (dotted bars, reference).

FIG. 2 shows the % increase in fibroblasts treated with 2×10:1, 2×10²:1, 2×10³:1, 2×10⁴:1, 2×10⁵:1, and 2×10⁶:1 eq/eq alginate-spermidine complexes at 24 h (light shaded) and 48 h (darker shaded) vs untreated cell; first 2 bars from alginate-spermidine 2:1 eq/eq (stripped bars); last 2 bars from pure alginate 0.1% w/v (dotted bars, ref.).

FIG. 3 shows the % increase treated by 3×10:1, 3×10²:1, 3×10³:1, 3×10⁴:1, 3×10⁵:1, and 3×10⁶:1 eq/eq polymaleate-spermidine complexes with the same meanings of FIGS. 1 and 2.

FIG. 4 shows the % fibroblast increase treated with Spermidine 3HCl at serial dilution.

FIG. 5 shows the differences in % fibroblasts increase by supramolecular complexes minus the values by spermidine 3HCl alone at equivalent concentrations.

FIG. 6 plots the potency ratio of the supramolecular complexes compared to spermidine 3HCl at equivalent concentrations, i.e. expressed as ratio of % increases:% increases in fibroblast increase.

FIG. 7 shows the histologic section of a reconstituted human vaginal mucosa (RHVE) treated for 24 h with a 0.1% w/v aq. sol. of HA-spermidine 50:1 eq/eq after staining with the Ki-67 antigen; whilst FIG. 8 is the control tissue treated with 0.9% saline.

### DETAILED DESCRIPTION

The expression “supramolecular complex” as used herein describes a polyacid/polybasic complex formed by polyanionic polymer(s) and spermidine characterized by a ratio of anionic eq. (from polyanionic polymers) and cationic eq. (from spermidine) from about 10:1 to about 10⁷:1 eq/eq, more preferably from about 10²:1 to about 10⁴:1 eq/eq.

These supramolecular complexes possess a marked proliferative/regenerative activity that is significantly superior to spermidine, the polyanionic polymer, and the sum thereof. The supramolecular complexes are novels, as well as compositions comprising thereof for therapeutic/cosmetic purposes in need of fibroblast proliferation, as illustrated hereafter.

Said supramolecular complexes are characterized by a ratio≧10:1 e/eq, in that differing from complexes of polyanionic polymer and spermidine approaching equimolarity both by their structural features and by a significantly higher proliferative activity on fibroblasts.

The expression “polyanionic polymer and spermidine approaching equimolarity” as used herein means complexes having ratios from about 1:3 to 10:1 eq/eq of anionic equivalents from the polyanionic polymer to cationic equivalents from spermidine.

The expression “polyanionic polymer” refers, in the broadest sense understood in the art, to a polymeric material or polymer comprising a plurality of several anionic moieties per molecule. It includes natural/semi-synthetic polymers, and fully synthetic polymers containing a plurality of anionic moieties such as carboxylic (—COO⁻), sulphate (—OSO₃⁻), sulfonate (—SO₃⁻), phosphate (—OPO₃²⁻), phosphonates (—PO₃²⁻), and combination thereof.

The expression “polyanionic polymer” includes “polyanionically-derivatised polymer”, meaning previously non-polyanionic polymers being converted into polyanionic polymer with suitable derivatizating reactants. Examples of derivatizations are carboxymethylation, succinylation, or maleylation for carboxy groups; sulfation/sulfonation/sulfinilation for sulfate/sulfonate groups; phosphation/phosphorylation for phosphate/phosphonate groups.

While not intending to limit the scope of the invention in any way, typical useful polyanionic polymers include anionic phyto-polysaccharides, phyco-polysaccharides, and endopolysaccharides; semi-synthetic, and fully synthetic polyanionic polymers.

Natural polyanionic polymers may be phyto- and phyco-polysaccharides such as alginates, agar, gellan gum, ghatti gum, karaya gum, tragacanth gum, wellan gum, xanthan gum, κ- ι-, and λ-carrageenan, xylomannan sulfate, fucoidan, and fucogalactan,

Other natural polyanionic polymer may be the endopolysaccharides such as hyaluronate, cross-linked hyaluronate, and other glycosoaminoglycan like heparin, supersulfated and modified heparins, e.g. supersulfated heparin, fraxiparin, fondaparin, idraparin, chondroitin sulfate A, B, and C, and the K5 derivatives.

Suitable hyaluronate, alias hyaluranan (HA) may be either of animal or microbial origin, with molecular weight (MW) in ranging from 5,000 kDa to 10 MDa.

Semi-synthetic polyanionic polymer may be carboxymethylated polysaccharides such as carboxymethyl cellulose, carboxymethyl starch, carboxymethyl dextran, carboxymethyl chitosan, carboxymethyl chitins; sulphated polysaccharides such as rhamnan sulfate, dextran sulfate, cellulose sulfate, sulfochitosans, curdlan sulfate, glyloid sulfate (GP4324), carob gum sulfate (GP4327), pentosan polysulfate (PPS); and phosphated polysaccharides such as phosphocellulose, and phosphochitosan.

Semi-synthetic polyanionic polymer may also be HA derivatives, e.g. the Fidia HA derivatives; or the thiolated polysaccharides such as thiolated cellulose, thiolated alginates, thiolated chitosan, thiolated hyaluronate from ThioMatrix-Green River Polymers GmbH (Insbruck, Austria), and the like.

Synthetic polyanionic polymer may be polyacrylates and polymethacrilates, linear and cross-linked homopolymers and copolymers thereof such as acrylates/acrilamides, acrylates/alchyl C10-C30 acrylates, acrylates/octylacrylamides; Carbopol™, Carbomer™ and Pemulen™ from Noveon-Lubrizol, butyl-polyacrylic acid, poly(acrylate-co-acrylamidomethylpropane sulfonate), poly(acrylate-co-vinylsulfonate), and poly(acrylate-co-vinylbenzenesulfonate) copolymers; methylvinylethere/maleic and other maleate copolymers, alias “polymaleates”, e.g. of Gantrez™ type (ISP Corp.); and furthers as per US2010172861 or US2003021793; as well as poly(sodium 4-styrene sulfonate), Y-ART-4, suramin, thiolated carbomers, thiolated poly(met)acrylic acid, and the like.

The expression “polyanionic polymer” also encompasses anionic inorganic polymers such as polyphosphates; or recombinant polymers as disclosed in WO2002/077036.

Preferred polyanionic polymers for the inventive purposes are linear and crosslinked hyaluronates, alginates, linear and crosslinked polyacrylates, and polymaleates.

The polyanionic polymer to be used according to this invention has a molecular weight≧5000 Da; and/or an anionic density from 0.1 to 18 meq/g, preferably from 1 to 14 meq/g.

For the inventive purposes spermidine, N-(3-aminopropyl)-1,4-butanediamine, or “Spd” hereafter, is the substance of formula NH₂(CH₂)₃NH(CH₂)₄NH₂ as such or salt thereof may be used preferably of synthetic origin and, also preferably, of purity≧99% on dry basis.

The inventive supramolecular complexes are produced by acid-base exchange, e.g. by combining a polyanionic polymer salt (typically Na) with spermidine salt (e.g. 3HCl); or by combining polyanionic polymers in the acid form with spermidine as free base.

The remaining anionic equivalents in the polyanionic polymer within the complex may be in acid form or partially neutralized, e.g. with alkaline or earth-alkaline ions such as Na, K, Li, Ca, and Mg; or amines such as NH₄⁺, mono- di- and triethanolamine, tromethamine, isopropylamine, lysine, etherocyclic amines such as piperazine, and the like.

The combination of polyanionic polymer with spermidine may be carried out in solvated status, preferably in water and/or water-soluble solvents such as lower alcohols, etc. The so-formed supramolecular complex may be dried up, e.g. by liophylization, spray-drying, vacuum drying, and the like; or used as such in solution form, provided that the solvents were physiologically acceptable and compatible with the desired end-use.

In an embodiment, supramolecular complex are formed by combining one or more polyanionic polymer with spermidine directly within the tank-mixer used in the galenic manufacturing. It can be performed, e.g., by mixing the aqueous solutions of the reactants, optionally along with selected co-solvents, excipients, diluents, carriers, and adjuvants.

In alterative, the supramolecular complex according to the invention are prepared directly in solid state, e.g. by spraying a spermidine solution onto the polymer or, alternatively by blending or grinding the components in dry or partially wet forms, and the like procedures

Notwithstanding the applied procedure, the obtained supramolecular complexes may be used in admixture with several further excipients, diluents, carriers and adjuvants to produce a composition suitable for tissue repair and maintenance.

The inventive supramolecular complexes exhibited a high regenerative activity on fibroblasts, and progenitors thereof, hence being suitable for the preparation of medicinal and cosmetic composition with regenerative/reparative purposes.

In accordance with an aspect of the present invention, there are provided compositions and methods to regenerate connective tissues by eliciting/enhancing cell proliferation.

The term “connective tissues” or “CT” as used herein encompasses both “Proper CT” and “Special CT”, the latter include subtypes such as cartilage, bone, and adipose tissue.

Another object of invention is a medicinal or cosmetic composition comprising said supramolecular complex to regenerate or repair connective and fibroblast-based tissues. In several differentiated embodiments, the inventive compositions are administered to improve senescent tissues or to repair damaged tissues, including:
vaginal mucosa, i.e. in gynaecology, to treat vaginal atrophy and related disorders such as vaginal dryness and dyspareunia;
urethral mucosa, i.e. in urology, to treat interstitial cystitis in females or males (prostatitis), urethral fistula, and induced cystitis;
eyes, i.e. in ophthalmology, to treat cornea damage and keratoconjunctivitis sicca (dry eyes);
ear, throat and nose, i.e. in otolaryngology, to treat otitis, damaged eardrum, pharyngitis, dysphagia, nasal sinuses post-surgery, and epistaxis;
oral mucosa, i.e. in stomatology, to treat stomatitis, aphtous ulcer, and xerostomia, dry mouth and/or Sjogren's syndrome;
gingiva and periodontum, i.e. in periodontology, to treat gingivitis/periodontitis;
gastroduodenal, intestinal, and proctorectal mucosae, i.e. in gastroenterology, to treat peptic ulcer, gastrointestinal fistula, diverticulitis, coloproctitis, and rhagades;
senescent skin and adnexa, i.e. in cosmetic and dermatology, to treat wrinkles, stretch marks, skin wounds, and senescent alopecia;
dermal and hypodermal layers, i.e. in aesthetic medicine, to treat aesthetic defects by dermal fillers;
synovial fluids and joint tissues, i.e. in osteology, to treat ostheoarthritis and joint damage;
alveolar lungs, i.e. in pneumology, to treat emphysema, respiratory distress syndrome, and bronchopulmonary dysplasia in premature infants.

The compositions of invention will comprise supramolecular complexes of 10³-10⁷ eq/eq ratio in amount from about 0.01% to about 10% w/w; and supramolecular complexes of 10-10² eq/eq ratio from about 0.0001% to about 10% w/w of the composition.

An inventive composition are preferably designed to provide an amount of spermidine from about 10 μmol to 0.1 nmol per unit dose, more preferably from about 1 μmol to 1 nmol per unit dose, even more preferably from about 100 nmol to 10 nmol per unit dose.

Low dosage levels are recommended in leave-in compositions, wherein a long period of time is sufficient to release of a therapeutic amount of spermidine onto the target tissue. The upper dosage levels are instead recommended in rinse-off or other composition with short-time contact to provide a sufficient level of spermidine to elicit tissue regeneration. The amount and duration of treatment shall be determined according to the target and patient condition, typically over a period from 30 to 60 days or more until relief is achieved, than it may be ceased, tapered, or reduced for an indefinite period.

Noteworthy, the composition of invention differs from compositions, if any, occasionally comprising polyanionic polymer(s) and spermidine that were separately admixed thereto and thus may not, or just partially do, produce the corresponding supramolecular complex.

The composition of invention may be produced according to known techniques with physiologically acceptable ingredients and carriers in order to afford the better benefit/risk profile, e.g. those listed in INCI-CTFA Annex 93/35/ECC and, or in Pharmacopoeias.

A fluid composition may have different presentations, including gel, lipogel, aerosol, spray, lotion, milk, foam, cream, W/O, O/W or multi-phase emulsions, mucoadhesive patches and so on, along with suitable excipients, carriers, or propellants.

The compositions for use in gynaecology may be conceived in form of suppository, ovules, vaginal tablets, disposable applicators, pessary, suppository, tampon, implant such as a ring, etc. These compositions can be packaged in a vaginal applicators such as disposable applicators (Lameplast, Italy) or squeezable long-necked tubes as 5 ml-Geliofil vaginal gel sold by Effik SA (Meudon-La-Fort, France), spray, foams, and the like.

The compositions for use in urology may be conceived as instillation device containing sterile solutions of the HA- and/or chondroitin sulfate-spermidine complexes.

The compositions for use in ophthalmology must be compatible with ocular tissues, e.g. sterile solution drops in multi-dose or as single use, disposable packaging.

The compositions for oral mucosae may be formulated in many ways, e.g., as per ADA/PDR: Guide to Dental Therapeutics, 4th Ed.′ such as mouthwash, oral solution, spray, gel on film, dentifrices, tooth powder, dental tablets, cream and gels, chewing gum, chewable tablets and lozenges, gel, film, gel-on-film, granules, paste, spreadable powders, etc. for application on oral cavity directly or by an external device.

The compositions for use as dermal fillers or in viscosupplementation shall be packaged as a sterile device. The dermal filler will have a capacity from 0.5 to 2 ml and thin needle, e.g. 30 G, 31 G and 33 G; while the viscosupplementation device will be filled in syringes or vials of 0.5-50 ml, preferably of 1-10 ml coupled with larger needle e.g. 27 G.

Preferred supramolecular complexes for such application are preferably formed with cross-linked HA (HAxx) and spermidine at final concentration from 0.2 to 2% w/w in buffered sterile water, with viscosity ranging from 500 to 8000 cP.

Complexes for use as aesthetic filler may be prepared by reacting spermidine or salt thereof with a HAxx prepared ad hoc, or with a commercially available HAxx.

Examples of commercial HAxx in aesthetic medicine are Artz/Supartz, Healon, Hyalgan, Ostenil, Viscoseal, Orthovisc, Elevess, Adant, Arthrum H, Hyalart, Hyalubrix, Replasyn, Synvisc Hylan G-F 20, Durolane, BioHy, Arthrease, or Orthovisc; in viscosupplementation are Juvelift; Restilane, Restylane touch, Perlane, Juvederm, Hyalaform, Rofilan, Prevelle, Puragen, Macrolane, Elevess, Belotero, and Teosyal.

An ad hoc preparation of HAxx consists in reacting a linear HA with a cross-linking agent, e.g. BDDE (butanediol diglycidyl ether), DVS (divinyl sulphone), DEO (1,2,7,8 diepoxyoctane), or BCDI (p-phenylene bisetheyl carbodiimide), under moderate heating. The obtained mass is further processed, i.e. washed, diluted, buffered and packaged into a single-use syringe, finally sterilized by steam cycles or γ-rays.

Dermal fillers may be applied in lips, folders, face contour remodelling, chicks and chin surgery, wrinkles, glabellar wrinkles and oral commissure, crow's feet, post acneic and traumatic wounds, soft tissues issues, breast and body remodelling, or rhinoplasty sequels.

The sterile device for injection according may also comprises auxiliary agents such as anestetics, analgesics, biocides, antivirals, muscle relaxants, salts, buffer, and diluents.

Gynecologic compositions may include a sexual hormone such as low-dose estrogens, estro-androgenic precursors, androgens, and selective estrogen receptor modulators (SERM). Exemplary estrogens are 17β-estradiol and esters, 17α-estradiol and esters, estriol and esters, estrone and esters, estrone sulfate, conjugated estrogens such as Premarin™ (Wyeth-Ayerst) and Cenestin™ (Duramed) or esterified like Estratab™ (Solvay), equilin and esters, 17α-ethynilestradiol and esters. Other estrogens include estrofurate, quinestrol, mestranol, and phytoestrogens like equol and enterolactone. A preferred estrogen is 17β-estradiol from 1 to 25 μg (topical) and from 1 to 0.1 mg (oral). Exemplary estro-androgenic precursors are dehydroepiandrosterone (DHEA) and 5-androstenediol, e.g. DHEA 2.5-100 mg. Exemplary androgens are androstenedione, androst-5-ene-3β,17α-diol, 4-androstenediol, androsterone, epitestosterone, testosterone, and metandriol.

The composition inventive may include an anti-infective agent including antibiotics, anti-fungals, antivirals, biocides etc. Several additional ingredients may added to the inventive compositions. Examples include, but not limited to, anti-inflammatories, immuno-suppressant, buffering agents, plant or algal extracts, antihistamines, antioxidants, astringents, fragrances, dyes, vitamins, sunscreens, deodorants, preservatives, and other customary ingredients. Other inventive compositions may similarly contain a variety of complementary ingredients as those skilled in the art can select using conventional criteria.

### EXAMPLES

### Examples 1-6 and Comparative Example 1

### Supramolecular Complexes HA-Spd and Fibroblast Proliferation

Supramolecular complexes HA-Spd with ratio of anionic equivalents (HA ˜3 meq/g) and anionic equivalents from Spd (3 eq./mol) were prepared by reacting Spd.3HCl (Sigma-Aldrich) as aq. solution with sodium HA, ophtalmic grade (Bioiberica, Barcelona, Spain) to afford a series of 10:1, 10²:1, 10³:1, 10⁴:1, 10⁵:1 eq/eq complexes at 0.1% w/v in water.

These supramolecular complexes were tested to evaluate the stimulatory activity on human fibroblasts (ATCC-CRL-2703). Culture media with tested samples at 0.1% w/v were added to the wells containing cells in the G0 phase of cell cycle. Cells were exposed to each complex for 24 and 48 hours (medium was replaced every 24 h) at 37° C. and 5% CO₂. At the end of incubation period, MTT coloration was performed in order to evaluate cell viability and the increasing proliferating rate compared to untreated control cells. For each determination 6 tests were carried out. After exposure, cells were washed with 200 ml of PBS, then 200 μl of MTT-medium are added to each culture well and incubated for 4 hours at 37° C. and 5% CO₂. MTT medium is then removed and 200 μl of MTT dissolving solution (10% Triton X-100 and 0.1 N HCl in dry isopropanol) were added. After shaking on a rotating plate for 20-30′ to dissolve crystals and homogenize the solution, absorbance was read at 570 nm; background reading at 690 nm.

Results as % cell proliferation vs untreated (control) cells are illustrated in Table I below.

TABLE I

| | |
|---|---|
| HA-Spd complex ratio | % Proliferative increase |

| (anionic eq./cationic eq.) | at 24 h | at 48 h |
|---|---|---|
| ~1:1 (comparative) | 3.94 | 8.48 |
| ~10:1 | 19.21 | 24.48 |
| ~10²:1 | 44.66 | 45.68 |
| ~10³:1 | 60.43 | 62.87 |
| ~10⁴:1 | 79.47 | 80.65 |
| ~10⁵:1 | 73.57 | 84.30 |
| ~10⁶:1 | 38.26 | 43.97 |
| HA 0.1% (reference, no Spd) | 5.42 | 5.85 |

The data clearly show that the supramolecular associations between HA and spermidine are a mean 1 log higher compared to HA, a well-known cell growth promoter.

Moreover, the mitotic potency of Spd-HA complexes is significantly higher then Spd alone, which in turns has a bimodal pattern (see Comparative Example 4).

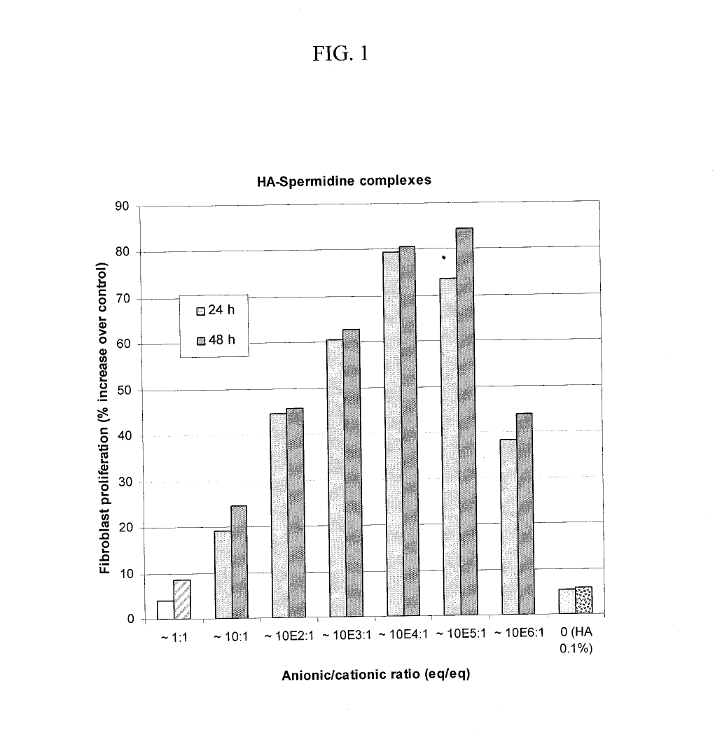

The results are plotted in FIG. 1 as % increase after treatment with HA-Spd complexes at 24 h (light shaded) and 48 h (darker shaded) vs untreated cell; first 2 bars being the equimolar HA-Spd complex (stripped bars, outside the invention); last 2 bars being pure HA 0.1% w/v (dotted bars, reference).

### Examples 7-12 and Comparative Example 2

### Supramolecular Complexes Alginate-Spd and Fibroblast Proliferation

Supramolecular complexes were obtained from Na alginate, Satialgine S 1100 (Cargill) and Spd 3HCl aq. sol. affording alginate-Spd complexes at 0.1 w/v. Said complexes were tested with the method described in Examples 1-6. The results are illustrated in Table II.

TABLE II

| | |
|---|---|
| Alginate-Spd complex ratio | % Proliferative increase |

| (anionic eq./cationic eq.) | at 24 h | at 48 h |
|---|---|---|
| 2:1 (comparative) | 6.23 | 3.29 |
| 2 × 10:1 | 24.41 | 17.42 |
| 2 × 10²:1 | 37.88 | 28.42 |
| 2 × 10³:1 | 61.95 | 52.86 |
| 2 × 10⁴:1 | 74.24 | 54.59 |
| 2 × 10⁵:1 | 60.77 | 46.71 |
| 2 × 10⁶:1 | 41.75 | 41.46 |
| Alginate 0.1% (ref., no Spd) | 9.60 | 7.28 |

The data illustrates a good proliferative potency of the alginate-Spd complexes, which can be usefully applied in the repair and maintenance of damaged or senescent connective.

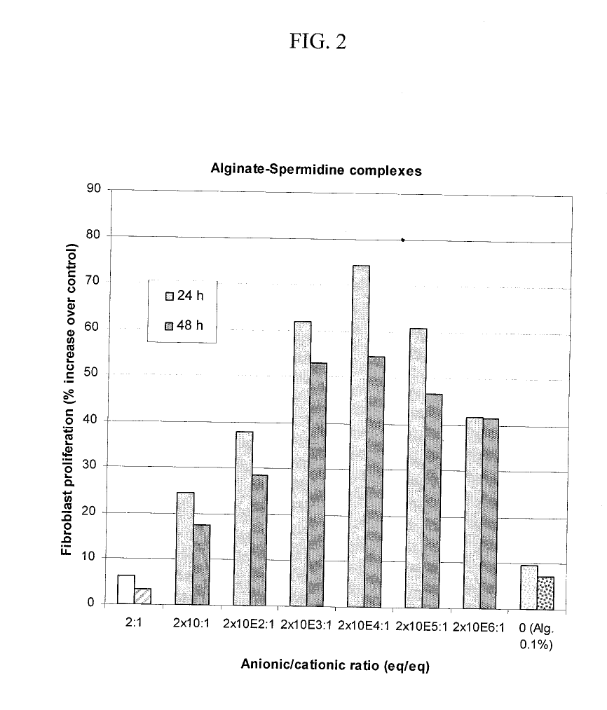

The results are plotted in FIG. 2 as % increase of fibroblasts treated with the alginate-Spd complexes, 0.1% w/v, at 24 h and 48 h vs untreated cell; first 2 bars being alginate-Spd 2:1 eq/eq complex (outside the invention); last 2 bars being alginate 0.1% w/v (dotted bars).

### Examples 13-18 and Comparative Example 3

### Supramolecular Complexes Polymaleate-Spd and Fibroblast Proliferation

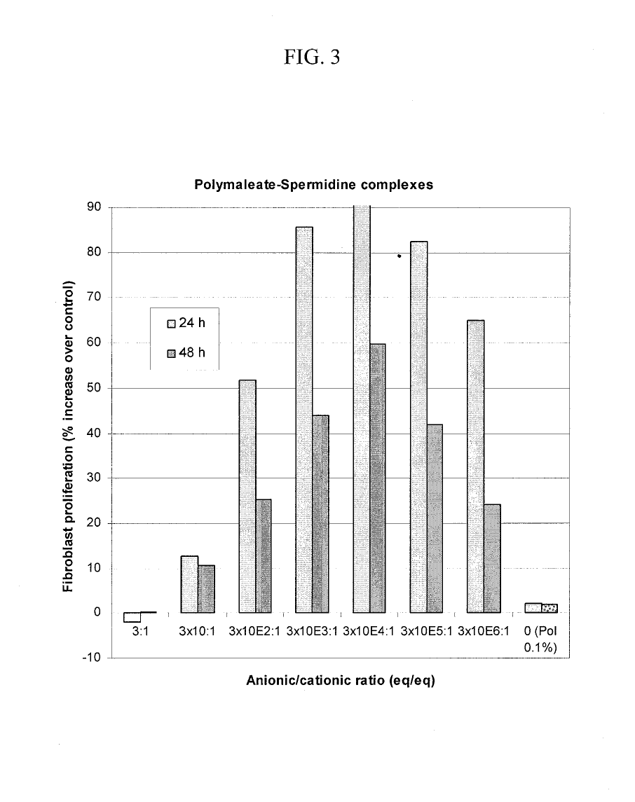

Complexes were obtained as polymaleate-Spd from the PVM/MA copolymer by neutralizing Gantrez S97BF (ISP Corp.) with 8-9 ml of NaOH 1N per g, then reacting with Spd HCl and completing with water to 0.1% w/v. The tests were carried out with the method outlined in Examples 1-6. The results are illustrated in Table III and by FIG. 3.

TABLE III

| | |
|---|---|
| Polymaletate-Spd complex ratio | % Proliferative increase |

| (anionic eq./cationic eq.) | At 24 h | at 48 h |
|---|---|---|
| 3 × 10:1 (comparative) | 12.63 | 10.49 |
| 3 × 10²:1 | 51.68 | 25.22 |
| 3 × 10³:1 | 85.69 | 44.02 |
| 3 × 10⁴:1 | 91.75 | 59.71 |
| 3 × 10⁵:1 | 82.49 | 42.03 |
| 3 × 10⁶:1 | 65.15 | 24.00 |
| Polymaleate 0.1% (ref., no Spd) | 2.19 | 1.99 |

The activity of the polymaleate-Spd complex is the highest among tested complexes, with a peculiar superiority in longer (48 h) compared to short (24 h) time of contact.

### Comparative Example 4

### Spermidine in Fibroblast Proliferation and Comparison with the Supramolecular Complexes

The fibroblast proliferation test was carried out at with Spd 3HCl at serial dilution, as shown hereafter, by the method illustrated in Examples 1-6. Results are shown in Table IV.

TABLE IV

| | |
|---|---|
| Spermidine (Spd) 3HCl | % Proliferative increase |

| | | |
|---|---|---|
| concentration | at 24 h | at 48 h |

| | | | |
|---|---|---|---|
| 10 | mM | −9.85 | −9.5 |
| 1 | mM | −0.16 | −3.22 |
| 100 | μM | 1.31 | 4.09 |
| 10 | μM | 2.3 | 8.48 |
| 1 | μM | 7.55 | 13.01 |
| 100 | nM | 26.27 | 29.39 |
| 10 | nM | 31.86 | 35.96 |

The pure substance (outside invention) has a bimodal pattern: a suppressive effect at mmolar concentration, and a positive effect at μmolar or nmolar levels. Although this dual mode was somehow expected, the significant lower potency versus the complexes was not.

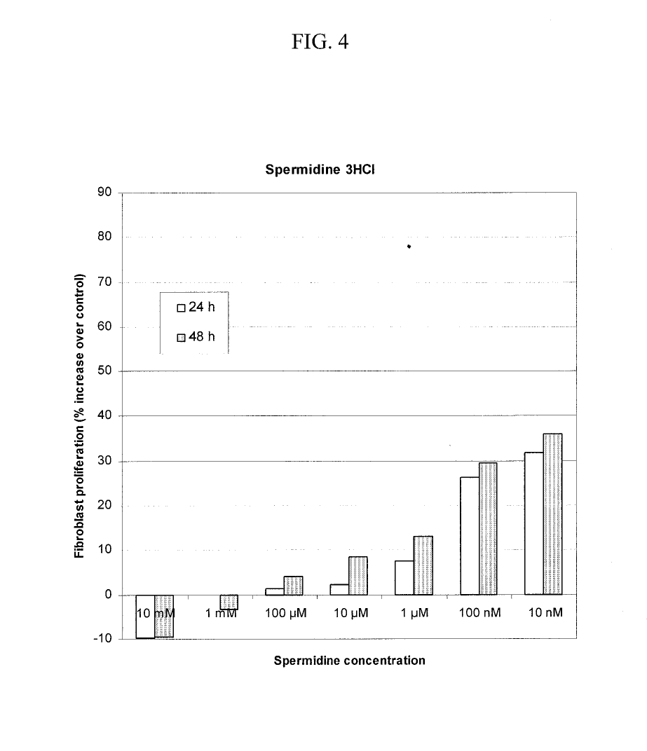

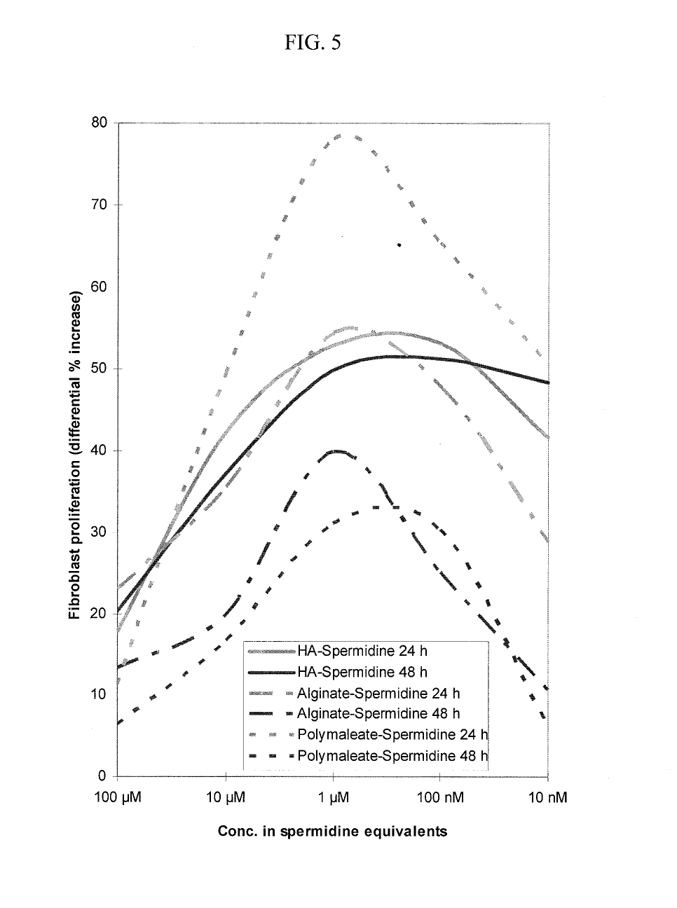

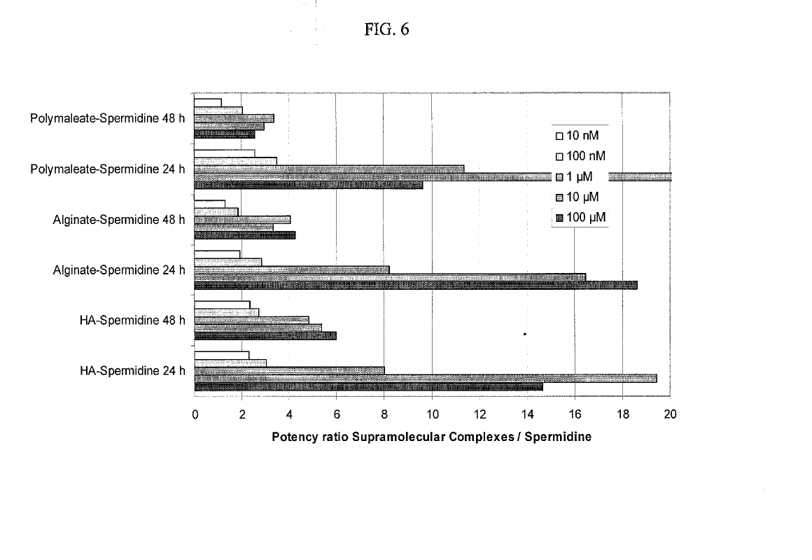

Results are displayed in FIG. 4, while FIG. 5 shows the net differences in % increase of the complexes minus the value of Spd 3HCl at equivalent conc. Finally, FIG. 6 plots the potency ratio as % /% ratio increase of supramolecular complex to spermidine alone.

### Example 19

### Supramolecular Complex HA-Spd 50:1 eq/eq and Gene Proliferation on Engineered Mucosa

The Ki-67 protein is a cellular marker for proliferation associated with cell proliferation. During interphase, the Ki-67 antigen can be exclusively detected within the cell nucleus, whereas in mitosis most of this protein is relocated chromosomes surfaces. Ki-67 is present during all active phases (G1, S, G2, and mitosis), but is absent in resting cells (G0).

The test uses Reconstituted Human Vaginal Epitelium (RHVE) from SkinEthic™ (Lyon, France), i.e. an immortalized human cell line (A 431) that was cultivated for 5 days onto a polycarbonate inert filter on air/liquid interface in a defined culture medium.

The proliferative stimulation on RHVE exposed to HA-Spd 50:1 eq./eq. for 24 hours at 37° C. and 5% CO₂ was evaluated. The control was sodium chloride 0.9%. Specimen were then treated with Ki.67 antibody overnight at 1:100 concentration, with the Ki.67 detected by Superpicture Polymer Detection kit (Invitrogen), with HRP and DAB as chromogens.

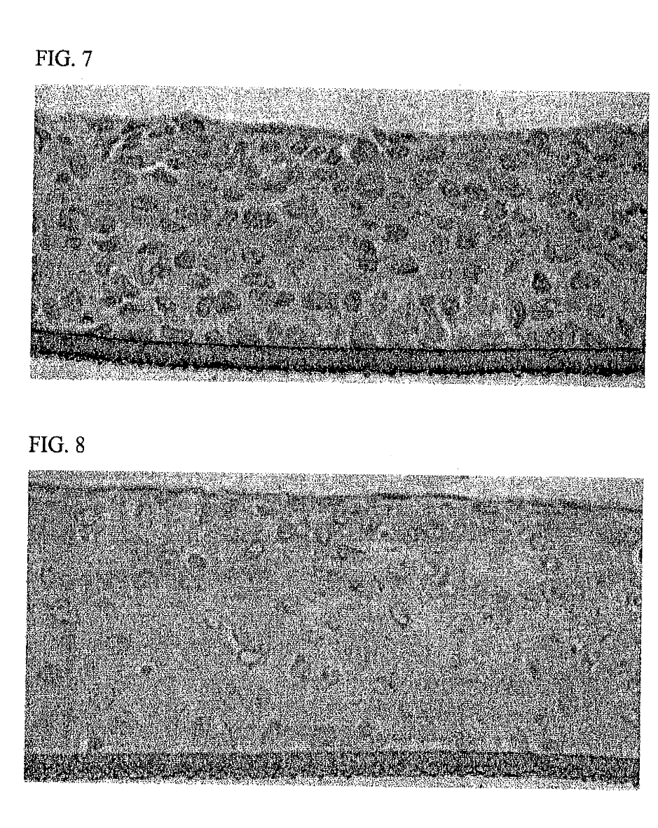

Final results are depicted in FIG. 7 indicates an activation signal of proliferative phase, wherein several KI.67 positive cells located on basal, suprabasal and superficial layers are noted. FIG. 8 is the control (0.9% saline) showing few activated cells at the apical level.

### Example 20

### Metabolic Pattern of HA-Spd 50:1 eq/eq in Native Gingival Fibroblasts

Tissue specimens of non-inflamed periodontal gingival were obtained from the premolar area during oral surgery (six females, 20-30 years old). Each biopsy was washed with 0.1 M D-PBS and immediately minced with sterile scissors. Tissue fragments were transferred to 25 cm2 Nunc flasks and, after adherence, supplemented with 5 ml DMEM containing 10% BFS 100 UI/ml penicillin, 10 ng/ml streptomycin, and 25 μg/ml amphotericin B. Cultures were maintained in humidity saturated atmosphere (5% CO₂, 37° C.) and routinely subcultured after use of 0.1% trypsin 0.02% EDTA for cell release. At given times cell culture supernatants were collected and fibroblasts washed in PBS, trypsinized and harvested by centrifugation (100×g, 5 min). Treatment: 4 doses, 1 time point (24 or 48 h) with sample treated with HA-Spd 10⁴:1, whilst untreated specimen served as control.

mRNA Levels for TGF-β1, LH2B, TIMP-1, TIMP-2 and GAPDH by Real-Time RT-PCR

Total RNA was isolated by a modification of the acid guanidinium thiocyanate-phenol-chloroform method (Tri-Reagent, Sigma). 1 μg of total RNA was reverse-transcribed in 20 μl final volume of reaction mix (Biorad). The primers sequences for the target genes was Beacon Designer 6.0 Software (BioRad). GAPDH was used to normalize for differences in amount of total RNA in each sample. Amplification was conducted in a final volume of 20 μl per well with 10 μl of 1× SYBR Green Supermix (BioRad), 2 μl of template, 300 μmol of each primer, each sample analyzed in triplicate in duplicate amplifications. The cycle threshold and gene expression levels relative to GAPDH were calculated by 2^{-ΔΔCt} method.

### COL-I and COL-III Protein Levels by Slot Blot.

To access both COL-I and COL-III secreted by palate and tuberosity fibroblasts, cell culture media was concentrated 20-fold with Centricon 10 columns (Amicon Y10, Millipore). Protein content was determined by a colorimetric assay (DC Protein Assay, Bio Rad); 100 μg of total protein per sample in final vol. of 200 μl of Tris buffer saline (TBS) was spotted onto a nitrocellulose membrane in a Bio-Dot SF apparatus (Bio-Rad). Membranes were blocked for 1 h with 5% skimmed milk in TBST (TBS containing 0.05% tween-20), pH 8, and incubated for 1 h at room temperature in monoclonal antibody to COL-I (1:1000 in TBST) (Sigma) or to COL-III (1:2000 in TBST) (Sigma). After washing, membranes were incubated in HRP-conjugated rabbit anti-mouse serum (1:80,000 in TBST) (Sigma) for 1 h. Immunoreactive bands were Amplified Opti-4CN substrate (Bio Rad) and scanned (UVBand, Eppendorf).

### MMP-1 Protein Levels and Activity by Western Blot.

Concentrated culture media (5 μg of total proteins) was diluted in SDS-sample buffer, loaded on 10% SDS-polyacrylamide gel, separated under reducing and denaturing conditions at 80 V, and transferred at 90 V to a nitrocellulose membrane in 0.025 M Tris, 192 mM glycine, 20% methanol, pH 8.3. After electroblotting, the membranes were air dried and blocked for 1 h. After washing, membranes were incubated for 1 h at room T in monoclonal antibody to MMP-1 (1 μg/ml in TBST, Oncogene Research) and, after washing, in HRP-conjugated rabbit anti-mouse serum (1:40000 dilution, Sigma-Aldrich). Immunoreactive bands were revealed by Amplified Opti-4CN substrate (Bio Rad) and scanned (UVBand, Eppendorf).

### Gelatinases (MMP-2 and MMP-9 Activity) by SDS-Zymodraphy.

Concentrated culture media was mixed 3:1 with sample buffer (containing 10% SDS). Samples (5 μg of total protein per sample) were run under non-reducing conditions without heat denaturation onto 10% SDS-PAGE co-polymerized with 1 mg/mL of type I gelatin. The gels were run at 4° C. After SDS-PAGE, the gels were washed twice in 2.5% Triton X-100 for 30′ each, incubated overnight in a buffer at 37° C. The MMP gelatinolytic activity was detected after staining the gels with Coomassie brilliant blue R250, as clear bands on a blue background.

### Fibroblast Count

The effect of proliferation was assessed by the cell ability to exclude trypan blue, performed according to Patterson M K Jr in: Jacob & Pastan, eds. Methods in Enzymology, vol. 58. New York: Academic Press; 1977:141.

Results reveal a complex pattern of regulatory activities of degradative enzymes such as collagenases and MMP; and modulation of TGF-β1, LH2B, TIMP-1/2 and GAPDH, which revert the previous finding of Gagliano N at al. J Periodontol 2005; 76:443-9.

### Example 21

### Simple HA-Spd Gel

10 g of sodium HA (MW 1.2 MDa) was dissolved in 800 ml of water until full hydration, then 10 ml of 1 mM Spd were added under stirring for 5′ to afford a HA-Spd 10³:1 eq/eq complex. Then 0.9 g of benzyl alcohol were admixed and stirred, and the final volume completed to 1 liter with purified water. The gel was loaded in 60-ml PE tubes suitable for most applications as uro-genital, oral, ear, nose, throat mucosae, skin, and so on.

### Examples 22-23

### CS/HA-Spd Sterile Solutions

A sterile solution was prepared with the ingredients as set forth in Table V below.

TABLE V

| Ingredient | Quantità (in 100 ml) | |
|---|---|---|
| Na chondroitin sulfate (CS, dry basis) | 1.00 | g |
| Sodium HA | 1.00 | g |
| Spermidine 3HCl, 1 mM | 20.0 | ml |
| Sodium chloride | 0.85 | g |
| Dibasic sodium phosphate 7H₂O | 42.0 | mg |
| Monobasic sodium phosphate 2H₂O | 4.00 | mg |

| | |
|---|---|
| Sterile water | to 100 ml |

NaCl and phosphates were dissolved in water. Sodium chondroitin sulphate (CS) and HA (6 meq) were added and mixed until full hydration, spermidine solution was then added to afford a ˜10³:1 eq/eq mixed complex. If necessary pH is adjusted to 7.2 with 1N NaOH or 1N H₃PO₄. Final volume is completed with sterile water for injection.

Sterile filling of 20 ml aliquots of the solution into Flint I type vials previously sterilized at 250° C. for 180′ with rubber stoppers is suitable for instillation in interstitial cystitis.

Another portion is sterile filled in 0.5-ml disposable PE-tubes suitable for ophtalmic use.

### Example 24

### Polymaleate-Spd Gel

A gel was prepared with the ingredients as set forth in Table VI below.

TABLE VI

| Ingredient | Quantity (in 100 ml) | |
|---|---|---|
| Gantrez S97BF | 1.00 | g |
| Spermidine 3HCl, 1 mM | 100 | μl |
| Propylen glycol | 15.0 | g |
| Parabens | 0.50 | g |

| | |
|---|---|
| Purified water | to 100 ml |

Gantrez S97BF, a polymaletate copolymer by ISP Corp (Wayne, N.J., USA) was dispersed in water and 8 ml 1N NaOH. Spermidine solution was added and stirred for 5′ to afford polymaleate-Spd 3×10⁴:1 eq/eq complex. The other components are admixed to end up with a consistent gel suitable for most applications cited herein.

### Example 25

### HA-Spd Gel

A gel was prepared with the ingredients as set forth in Table VII below.

TABLE VII

| Ingredient | Quantity (in 100 ml) | |
|---|---|---|
| Sodium HA | 1.20 | g |
| Spermidine 3HCl, 1 mM | 120 | μl |
| Xylitol | 7.50 | g |
| Dichlorobenzyl alcohol | 0.50 | g |
| Cu chlorophyl | 0.12 | mg |
| Arome | 0.30 | g |

| | |
|---|---|
| Purified Water | to 100 ml |

Sodium HA (MW 1.2 Md) was dissolved in water until full hydration. The spermidine solution was then added and stirred for 5′ to afford a HA-Spd 10⁴:1 eq/eq complex. The remaining ingredients were added and mixed to afford a multipurpose gel, e.g. indicated in stomatologic applications.

### EXAMPLE 26

### Mixed Polvanionic-Spd Gel

A gel was prepared with the ingredients as set forth in Table VIII below.

TABLE VIII

| Ingredient | Quantity (in 100 ml) | |
|---|---|---|
| Sodium HA | 0.20 | g |
| Sodium carboxymethylcellulose (CMC) | 2.50 | g |
| Polycarbofil | 0.30 | g |
| Spermidine 3HCl, 1 mM | 2.50 | ml |
| PEG-40 hydrogenated castor oil | 1.00 | g |
| Disodium EDTA | 0.05 | g |
| Xylitol | 7.50 | g |
| Dichlorobenzyl alcohol | 0.50 | g |
| Cu chlorophyll | 0.12 | mg |
| Favours | 0.30 | g |

| | |
|---|---|
| NaOH | to pH 6.5 |
| Purified water | to 100 ml |

Sodium HA, Blanose 7HXF (CMC), and Noveon AA-1 polycarbophil were dissolved in water until full hydration affording a viscous gel. The spermidine solution was then added and stirred for 15′. Remaining ingredients were singularly added and mixed to afford a homogenous green-coloured gel useful for mucosa repair.

### Examples 27-28

### Polymaleate-Spd Gel with NAC and Mucoadhesion Inhibitor

A gel was prepared with the ingredients as set forth in Table V below.

TABLE V

| Ingredient | Quantity (in 100 ml) | |
|---|---|---|
| Gantrez S97BF | 0.17 | g |
| Spermidine 3HCl, 1 mM | 500 | μl |
| Polaxamer 427 | 20.0 | g |
| PEG-40 hydrogenated castor oil | 1.00 | g |
| Povidone | 5.00 | g |
| Sodium saccharinate | 0.30 | g |
| Benzalkonium chloride | 0.10 | g |
| N-acetyl-cysteine (NAC) | 0.30 | g |
| d-Mannose | 4.00 | g |

| | |
|---|---|
| NaOH 1N | to pH 6 |
| Purified water | to 100 ml |

Polymaleate (Gantrez S97BF) was suspended in water and titrated with 1N NaOH until pH 6. Spermidine was then admixed to afford a polymaleate-Spd 10³:1 eq/eq complex.

The addition of next ingredients ended up with a homogeneous gel suitable for use on damaged mucosae having a ancillary biofilm disruptor activity.

An still enhanced formulation was conceived with the addition from 5% to 15% w/w of D-Mannose to afford the inhibition of bacterial adhesion onto the so-treated mucosae.

### Example 29

### High Mucoadhesive Thiolated PAA-Spd Gel

0.2 g of poly(acylic acid)-cysteine with thiol content of 0.5 meq/g (Green River Polymers GmbH; Insbruck, Austria) was suspended in 90 ml water and NaOH to pH 6.5. Then 40 μl Spermidine 3HCl, 1 mM were added and stirred for 15′ affording a consistent gel with high mucoadhesivity, e.g. particularly recommended in ophthalmology.

### Example 30

### Carbopol-Spd Modified Mouthwash

1 g of Carbopol Ultrez 20 (Noveon-Lubrisol) was dissolved in 500 ml of the commercial mouthwash Iodosan Antiplacca (Iodosan SpA, GSK group). The pH was corrected to 6.5 with 1N NaOH. Then 5 ml of 1 mM spermidine 3HCl were added and mixed until a homogeneous solution suitable for oral care, stomatology issues and gingivitis.

### Example 31

### HA-Spd Chewing-Gum

A blend comprising xylitol, sodium HA and spermidine at 100:10:1 w/w/w ratio obtained by dry grinding said components was supplied to Gum Base Co Srl (Lainate, Italy) with the instruction to manufacture of chewing gum with 0.8% of such blend. The resulting product is characterized by pleasant palatability usefully applied in gingival healing.

### EXAMPLE 32

### In Vivo Evaluation on Stomatitis (Case Study)

A 46-years female with recurrent apthous ulcer on mouth was given the gel of Example 21 and instructed to apply it at least twice a day. The subject referred a resolution in about a week against the 3-4 weeks generally needed. The product was well tolerated, except for the bitter taste, likely due to benzyl alcohol.

### Example 33

### Alginate-Spd Patches

Patches were prepared with the ingredients as set forth in Table IX below.

TABLE IX

| Ingredient | Quantity (in 100 ml) | |
|---|---|---|
| Spermidine 3HCl | 84.0 | mg |
| Sodium alginate | 27.4 | g |
| Propylen glycol | 4.70 | g |
| PEG 1000 | 9.50 | g |
| PEG 1500 | 10.4 | g |
| PEG 4000 | 38.2 | g |
| PEG 8000 | 3.80 | g |
| Eucalyptol | 0.50 | g |

Satialgine 1100 (Cargill), propylen glycol and PEG 1000 were slowly mixed to form a homogeneous mass. Spermidine was incorporated under mixing, then other components were added and melted to ≦80° C. Resulting alginate-Spd complex has 5×10²:1 eq/eq ratio. 150 mg of this mass were applied to patches of 7 mm×7 mm×1 mm in size and packaged in blister pack to protect it from light and air. Typical use of this patch are on wounded area on skin and mucosa.

### Example 34

### Carbopol-Spd Cream

A emulsion was prepared with the ingredients as set forth in Table X below.

TABLE X

| Ingredient | Quantity (in 100 ml) | |
|---|---|---|
| Spermidine 3HCl, 1 mM | 10.0 | ml |
| Carobopol Ultrez 20 | 0.40 | g |
| Cetostearyl alcohol | 7.00 | g |
| Ceteareth-6 | 1.50 | g |
| Ceteareth-25 | 1.50 | g |
| Liquid paraffin | 12.0 | g |
| Parabens | 0.10 | g |
| Propylen glycol | 8.00 | g |

| | |
|---|---|
| NaOH 1N | to pH 6.5 |
| Purified water | to 100 ml |

Carbopol, NaOH and the parabens were dissolved in water with a final pH of around 6.5. Spermidine 3HCl was added and stirred with propylen glycol to afford a Carbopol-Spd 1.8×10³:1 complex. The mix is heated to 80° C., the lipid ingredients separately mixed and heated to the same temperature, then the 2 phases were emulsionated under high speed.

### Example 35

### CMC-Spd Tablets

Tablets were prepared with the ingredients as set forth in Table XI below.

TABLE XI

| Ingredient | Quantità (in 100 g) | |
|---|---|---|
| Spermidine 3HCl | 0.80 | g |
| Sodium carboxymethyl cellulose (CMC) | 12.0 | g |
| Sodium HA (MW 10 kDa) | 10.0 | g |
| Na chondroitin sulfate (CS, dry) | 10.0 | g |
| Magensium stearate | 3.50 | g |
| Silica gel | 16.2 | g |

| | |
|---|---|
| Microcristalline cellulose | to 100 g |

Spermidine 3HCl was incorporated by wet-mixing with Blanose 7HXF (CMC), sodium HA, and chodroitin sulfate Na salt in a grinder with some drops of purified water. The next ingredients were admixed and the whole mixture was sieved at 50 mesh, the granules were compressed to afford tablets of ˜1 g each.

### Example 36

### CMC-Spd Gel Dentifrice

A toothpaste was prepared with the ingredients as set forth in Table XII below.

TABLE XII

| Ingredient | Quantity (in 100 g) | |
|---|---|---|
| Spermidine 3HCl, 1 mM | 2.00 | ml |
| Sodium carboxymethyl cellulose (CMC) | 0.80 | g |
| Xylitol | 1.00 | g |
| Propylen glycol | 7.50 | g |
| Sodium lauryl surcosinate | 1.70 | g |
| Macrogol 1600 | 1.00 | g |
| Aroma | 0.90 | g |
| Sodium saccharinate | 0.20 | g |
| Sodium phosphate | 0.20 | g |
| Dibasic sodium phosphate 7H₂O | 0.45 | g |
| Methylene blue, 1% | 0.10 | g |
| Parabens | 0.20 | g |

| | |
|---|---|
| Sorbitol, 70% | to 100 g |

The procedure follows the ordinary method for the production of a dentifrice, expect that CMC was first dissolved in water and added with the spermidine solution, then the remaining ingredients were incorporated to provide a blue translucent gel.

### Example 37

### Cross-Linked HA-Spd Complex (HAxx-Spd)

Synthesis of HAxx

The method is reproduced from WO 2011023355. In brief, 1.60 g of HA (MW 1.2 MDa) were dispersed in 20 ml of 0.25M NaOH solution of 75 μl of BDDE (Sigma-Aldrich); the mixture was heated to 42° C. and reacted for 2 hours. Then it was hydrated for 24 h with 20 ml of 1N HCl until neutral pH. The total volume was made up to 75 ml, then HAxx was precipitated with 2.5 vol. of ethanol, further washed with 75% ethanol until a specific conductivity of the eluate ≦30 pS/cm, and finally dried under vacuum at 40° C. to afford HAxx as white powder.

Preparation of HAxx-Spd Complex

The powder was hydrated with 0.9% w/v sterile saline solution, treated with dil. NaOH to pH 5.2 and then compounded with 2 peq. of spermidine 3HCl per g of solution.

### Example 38

### HAxx-Spd Dermal Filler

The gel obtained as in Example 37 was heated to 48° C., filtered at mesh 0.17 mm, then distributed in a 0.8 ml Schott TopPac™ (Schott North America Inc., Lebanon, Pa., USA.) syringe fitted with Tyco 30 G needle. The next sterilisation cycle with saturated steam at 121° C. for 10′ ended up with a sterile device suitable as dermal filler.

### Example 39

### HAxx-Spd Viscosupplementation Device

Same operation of Example 37 was carried out in a 5 ml Schott TopPac™ syringe fitted with Tyco 27 G needle, affording a sterile device suitable in osteoarthritis and joint repair.

### Example 40

### Non-Inferiority of HA-Spd vs Estradiol in Vaginal Atrophy

To compare Spd-HA and estrodiol in atrophic vaginitis, scores and end-points according to Ekin M et al. in Arch Gynecol Obstet. 2011; 283(3):539-43 were applied.

Postmenopausal women with symptoms of atrophic vaginitis were given vaginal tablets of 25 μg estradiol (n=21) (group I) or 2.5 ml of HA-Spd gel as per Example 1 supplied in 60 ml tubes coupled with 2.6 ml disposable vaginal applicators from Lameplast (n=21) (group II) and instructed to apply thereof every 2 days during 8 weeks.

Atrophic vaginitis were monitored by a 4-point scale of composite score of subjective symptoms; degree of epithelial atrophy determined as, none, mild, moderate and severe. Vaginal pH and maturation index were measured and compared in both groups.

Early data show a decrease in subjective symptoms, epithelial atrophy, and vaginal pH in both groups after treatment. Vaginal maturation values also improved at both study groups, although higher in group I compared to group II.

The innovation entails the optimized use of spermidine as growth factor to efficaciously treat senescent or damaged tissues. It was accomplished by supramolecular complexes that provide the mitogenic release of spermidine to elicit a reproliferative effect on fibroblasts, progenitors, and related connective cells. The tissues are then activated into a self-repair mechanisms, providing the faster recovery toward a healthy condition.

It should be understood that the foregoing relates only to preferred embodiments and to applicative examples of the present invention and that numerous modifications or alterations may be made therein without departing from the spirit and the scope of the invention as set forth in the appended claims.

1. A method for the treatment of ostheoarthritis or joint damage, which comprises administering to a subject an effective amount of at least a supramolecular complex formed by at least a polyanionic polymer and spermidine with a ratio of anionic equivalents and cationic equivalents from 10:1 to 10⁷:1 eq/eq, where the components of said supramolecular complex are intimately admixed, without any covalent bond between them.

2. The method of claim 1, wherein said ratio is from 10²:1 to 10⁴:1 eq/eq.

3. The method of claim 1, wherein the polyanionic polymer is a natural phytopolysaccharide, phycopolysaccharide, or endopolysaccharide; a semi-synthetic derivatized polysaccharide; or mixture thereof.

4. The method of claim 1, wherein the phytopolysaccharide, phycopolysaccharide, or endo polysaccharide is selected from the group consisting of alginates, agar, gellan gum, ghatti gum, karaya gum, tragacanth gum, welan gum, xanthan gum, sodium chondroitin sulfate, and linear or crosslinked hyaluronate derivatives.

5. The method of claim 1, wherein the semi-synthetic derivatized polysaccharide is selected from the group consisting of carboxymethyl cellulose, crosscaramellose, carboxymethyl starch, carboxymethyl dextran, carboxymethyl chitosan, linear or cross-linked hyaluronate derivatives, rhamnan sulfate, dextran sulfate, cellulose sulfate, curdlan sulfate, and phosphochitosan.

6. The method of claim 1, wherein the polyanionic polymer is selected from linear and crosslinked hyaluronates, alginates.

7. The method of claim 2, wherein the polyanionic polymer is selected from linear and crosslinked hyaluronates, alginates, linear and crosslinked polyacrylates, and sodium chondroitin sulfate.

8. The method of claim 1, wherein the supramolecular complex is administered by local injection of a medicinal composition comprising a supramolecular complex of polyanionic polymer(s) and spermidine ≧10:1 eq/eq ratio comprising an amount from 1 to 0.001 μmol of equivalent spermidine.

9. The method of claim 2, wherein the supramolecular complex is administered by local injection of a medicinal composition comprising a supramolecular complex of polyanionic polymer(s) and spermidine ≧10:1 eq/eq ratio comprising an amount from 1 to 0.001 μmol of equivalent spermidine.

10. The method of claim 6, wherein the supramolecular complex is administered by local injection of a medicinal composition comprising a supramolecular complex of polyanionic polymer(s) and spermidine ≧10:1 eq/eq ratio comprising an amount from 1 to 0.001 μmol of equivalent spermidine.

11. The method of claim 7, wherein the supramolecular complex is administered by local injection of a medicinal composition comprising a supramolecular comprising an amount from 1 to 0.001 μmol of equivalent spermidine.
